(19) United States
(12) Patent Application Publication
(10) Pub. No.: US 2026/0202451 A1
(43) Pub. Date: Jul. 16, 2026

(54) METHODS FOR IDENTIFYING TURNING POINTS OF WAVE SIGNALS

(71) Applicant: Alpha Intelligence Manifolds, Inc., New Taipei City (TW)

(72) Inventor: CHIA-HUNG LIN, New Taipei City (TW)

(21) Appl. No.: 19/443,045

(22) Filed: Jan. 8, 2026

Related U.S. Application Data
(60) Provisional application No. 63/744,827, filed on Jan. 13, 2025.

(51) Int. Cl. G01R 23/00; A61B 8/00; A61B 8/08

(52) U.S. Cl. CPC G01R 23/00; A61B 8/0883; A61B 8/5223

(57) ABSTRACT

The present invention relates to a computer-implemented method to identify extremum locations of wave signals. The method comprises steps of obtaining a displacement series comprising displacement of each time frame of the wave signal, determining a total state series which maximizes a penalized total path as an optimized state series based on the displacement series, and determining positions of turning points of the wave signal based on the optimized state series. A transition penalty is introduced to the penalized total path to suppress false identification of noise-derived local extrema as turning points. A dynamic programming (DP) algorithm may be used to perform the computer-implemented method.

### RELATED APPLICATION

This application claims the benefit of the U.S. Provisional Application No. 63/744,827 filed on Jan. 13, 2025, titled “A VIDEO-BASED MULTI-CONTOUR LEARNING METHOD FOR CARDIAC IMAGE SEGMENTATION AND ANALYSIS,” which is incorporated herein by reference at its entirety.

### BACKGROUND OF THE INVENTION

### Field of the Invention

The present invention relates to a computer-implemented method for identifying turning points of wave signals, especially wave signals with noise.

### DESCRIPTION OF RELATED ART

Finding locations of extrema in peaks and troughs is a commonly used signal processing technique for wave signals in various fields. For example, it can be used to analyze light curves of variable stars or respiratory signals. It can also be used in photoplethysmography (PPG), electrocardiogram (EKG) or echocardiography.

In echocardiography, tracking changes of ventricular volume requires identifying end-systole (ES) and end diastole (ED) in each cardiac cycle. However, due to irregular temporal volume changes and signal noise, traditional methods such as peak finding and min/max finding methods frequently identify local maxima or minima instead of true extrema in one cardiac cycle, which make those methods difficult to reliably estimate the cardiac cycle and ES/ED frames.

Therefore, it is desirable to develop a new method to more accurately identify extremum locations of wave signals.

### SUMMARY OF THE INVENTION

To resolve the problems, the present invention provides a computer-implemented method to identify extremum locations of wave signals, which enables the program to detect phase transition of a signal with fewer required parameters compared to prior art. In other words, the new method disclosed in the present invention reduces the needs for parameter adjustments, allowing consistent performance across datasets. The new method detects extrema without needing prior knowledge of the cycle period, making it suitable for signals with irregular cycles. It also shows high noise tolerance and jitter tolerance, which maintains accurate detection even with small noise disturbances and ensures correct extremum identification.

The computer-implemented method comprises steps of: (1) obtaining a displacement series comprising displacement of each time frame of the wave signal; (2) based on the displacement series, determining a total state series which maximizes a penalized total path as an optimized state series; and (3) determining positions of turning points of the wave signal based on the optimized state series. In this method, the total state series comprises a state value describing a status of ascending or descending of the wave signal at each time frame, and the penalized total path is calculated based on the displacement series and the total state series. To suppress false identification of noise-derived local extrema as turning points, a penalty value is introduced to the penalized total path every time when the state value changes in the total state series. In one embodiment, the optimized state series is determined by a dynamic programming algorithm.

The penalty value may be a statistical measure which measures the spread or dispersion of the signal value. In one embodiment, the penalty value is a standard deviation of the wave signal. In one embodiment, the penalty value is an interquartile range of the wave signal. In one embodiment, the penalty value is an average absolute deviation of the wave signal. In yet another embodiment, the penalty value is a median absolute deviation of the wave signal.

In one embodiment, the identified turning points of the wave signal are a plurality of time frames where the state value changes in the optimized state series.

In a preferred embodiment, the penalized total path is calculated with a formula

$${\sum }_{t = 1}^{T} ( {V}_{diff} ( t ) \cdot s ( t ) - Z ( t ) ) ,$$

wherein:
T is a final time frame of the wave signal;
V_{diff}(t) is a current displacement, which is a value of the displacement series at time frame t;
s(t) is a current state value, which is a value of the total state series at time frame t, wherein the current state value is either 1 to represent a status of ascending or −1 to represent a status of descending; and
Z(t) is a transition penalty at time frame t, wherein Z(t)=the penalty value, if s(t)≠s(t−1) and Z(t)=0, if s(t)=s(t−1).

In one embodiment of the computer-implemented method, said determining the optimized state series comprises iteratively calculating one or more penalized temporal paths and identifying one or more sub-optimal temporal paths from the one or more penalized temporal paths at each time frame. In one embodiment, each of the one or more penalized temporal paths is calculated by adding a penalized individual path of a current time frame to a previous temporal path of a previous time frame, and in a preferred embodiment each of the one or more penalized temporal paths is calculated by adding a penalized individual path of a current time frame to a previous temporal path of a previous time frame.

In a preferred embodiment, the penalized individual path comprises a current displacement and a transition penalty. In one embodiment, the one or more sub-optimal temporal paths comprise a first current path with a status of ascending at the current time frame, and a second current path with a status of descending at the current time frame. And the first current path and the second current path may be calculated based on a first previous path, a second previous path and the penalized individual path, wherein the first previous path is the largest among the previous temporal path having a status of ascending at the previous time frame, and the second previous path is the largest among the previous temporal path having a status of descending at the previous time frame.

In one embodiment, said determining the optimized state series comprises iterative steps of: (1) for each time frame, obtaining the first previous path, the second previous path, and the current displacement; (2) calculating the first current path and the second current path based on the first previous path, the second previous path, and the current displacement; (3) recording a first previous state value corresponding to the first current path, and recording a second previous state value corresponding to the second current path; and (4) updating the first previous path with the first current path for use in next iterative step, and updating the second previous path with the second current path for use in next iterative step.

After the iterative steps of calculating the first current path and the second current path are performed for a final time frame, the method may further comprise selecting the larger of the first current path and the second current path as an optimized final path. Then, the optimized state series may be obtained by iteratively determining a previous state value from the final time frame to a first time frame, wherein the previous state value at each time frame is selected from the first previous state value and the second previous state value. The selected previous state values of all time frames are integrated as the optimized state series.

In some embodiments, the first previous path and the second previous path described above respectively correspond to a first previous state series and a second previous state series, wherein each of the first previous state series and the second previous state series comprises a state value describing a status of ascending or descending of the wave signal at each time frame from a first time frame to the previous time frame. In one embodiment, the first previous path equals to the previous temporal path of the previous time frame calculated based on the displacement series and the first previous state series, and the second previous path equals to the previous temporal path of the previous time frame calculated based on the displacement series and the second previous state series.

In one embodiment, said determining the optimized state series comprises iterative steps of: (1) for each time frame, obtaining the first previous path, the first previous state series, the second previous path, the second previous state series, and the current displacement; (2) calculating the first current path and the second current path based on the first previous path, the second previous path, and the current displacement; (3) updating the first previous path with the first current path, and updating the second previous path with the second current path; and (4) updating the first previous state series with a first current state series corresponding to the first current path, and updating the second previous state series with a second current state series corresponding to the second current path. In a preferred embodiment, the first current path is the larger of a first ascending path and a second ascending path, and the second current path is the larger of a first descending path and a second descending path, wherein: (1) the first ascending path is the first previous path plus the current displacement; (2) the second ascending path is the second previous path plus the current displacement and minus the penalty value; (3) the first descending path is the first previous path minus the current displacement and the penalty value; and (4) the second descending path is the second previous path minus the current displacement. And in one embodiment, the first previous state series and the second previous state series are updated based on the following rules: (1) if the first current path is the first ascending path, then an ascending state is added to the first previous state series as the first current state series; (2) if the first current path is the second ascending path, then an ascending state is added to the second previous state series as the first current state series; (3) if the second current path is the first descending path, then a descending state is added to the first previous state series as the second current state series; and (4) if the second current path is the second descending path, then a descending state is added to the second previous state series as the second current state series. After the iterative steps described above are performed at a final time frame, the method may further comprises selecting the larger of the first current path and the second current path as an optimized final path. Then, the optimized state series may be obtained by determining the total state series which corresponds to the optimized final path.

In one embodiment, each of the one or more penalized temporal paths is calculated based on a formula TP(t,s)=TP(t−1,s′)+P(t), wherein:
TP(t,s) is the penalized temporal path with a current state value s;
TP(t−1,s′) is the previous temporal path with a previous state value s′;
P(t)=V_{diff}(t)−s(t)−Z(t) is the penalized individual path, wherein:
V_{diff}(t) is the current displacement, which is a value of the displacement series at the current time frame t;
s(t) is the current state value, which is a value of the total state series at the current time frame t, wherein the current state value is either 1 to represent a status of ascending or −1 to represent a status of descending; and;
Z(t) is the transition penalty at time frame t, wherein Z(t)=the penalty value, if s(t)≠s(t−1) and Z(t)=0, if s(t)=s(t−1).

Other objectives, advantages and novel features of the invention will become more apparent from the following detailed description when taken in conjunction with the accompanying drawings.

### BRIEF DESCRIPTION OF THE DRAWINGS

FIG. 1 is a state transition diagram illustrating ascending (S+) and descending (S−) states and possible transitions. Each state transition from an ascending state to a descending state or from a descending state to an ascending state will incur a penalty 6.

FIG. 2 shows an example of state/phase transition identified by a dynamic programming (DP) algorithm performing new method disclosed in the present application. The top row is an original frequency modulated (FM) signal, and in the middle row noises are introduced into the original signal. The signal then is used for transition point detection by DP algorithm performing extremum identification method. The detected transition points are shown as dots on the curve in the middle row, and the phase (ascending/descending or on/off) changes are illustrated in the bottom row.

FIG. 3 shows an example of state/phase transition identified by a dynamic programming (DP) algorithm performing new method disclosed in the present application. The top row is an original chirp signal, and in the middle row noises are introduced into the original signal. The signal then is used for transition point detection by DP algorithm performing extremum identification method. The detected transition points are shown as dots on the curve in the middle row, and the phase (ascending/descending or on/off) changes are illustrated in the bottom row.

FIGS. 4A and 4B shows heart muscle's contraction and relaxation cycle over time, and the extrema tracked by the DP algorithm. The curve is the strain signal, and the tracked extrema (maxima and minima) are indicated by the marks v and A.

### DETAILED DESCRIPTION OF PREFERRED EMBODIMENTS

The terminology used in the description presented below is intended to be interpreted in its broadest reasonable manner, even though it is used in conjunction with a detailed description of certain specific embodiments of the technology. Certain terms may even be emphasized below; however, any terminology intended to be interpreted in any restricted manner will be specifically defined as such in this Detailed Description section.

The embodiments introduced below can be implemented by programmable circuitry programmed or configured by software and/or firmware, or entirely by special-purpose circuitry, or in a combination of such forms. Such special-purpose circuitry (if any) can be in the form of, for example, one or more application-specific integrated circuits (ASICs), programmable logic devices (PLDs), field-programmable gate arrays (FPGAs), graphics processing units (GPUs), etc.

In the present application, a computer-implemented method to identify extremum locations of wave signals is provided. In this method, an objective function derived from a wave signal is used for extremum location identification. The objective function has a “state” parameter related to the different states (e.g., ascending and descending) of the wave signal at each time frame. By choosing suitable state parameters which maximize the objective function and identifying the locations where the state parameter changes in the wave signal, extremum locations (i.e., turning points) and the corresponding signal values of the wave signal can then be identified.

### Objective Function

The goal of the objective function is to ensure identification of state transitions while maximizing the difference of signal value between an identified peak and its neighboring trough. To achieve this goal, a transition penalty is introduced to the objective function to prevent local extrema caused by noise from being incorrectly identified as true extrema in a wave period. The objective function resembles the cumulated path an imaginary point has traveled during signal recordation, except that a penalty value is subtracted from the cumulated path every time a state transition occurs. Because the objective value is penalized every time when a transition occurs, false identification of noise-derived local extrema as turning points is suppressed by the transition penalty.

In a preferred embodiment, the objective function has the form:

$$Objective = {\sum }_{t = 1}^{T} ( {v}_{diff} ( t ) \cdot s ( t ) - Z ( t ) ) . \qquad ( Eq . 1 )$$

In this function, V_{diff}(t)=V(t)−V(t−1), which is the difference of signal value between consecutive frames t−1 and t, or the current displacement between time frames t−1 and t. The time frame is counted from the first frame (t=1) to the last frame (t=T). s(t) describes the state in time frame t. If the wave signal in time frame t is in ascending state (i.e., the signal value goes up), s(t)=1; and if the wave signal is in descending state (i.e., the signal value goes down), s(t)=−1. Z(t) is the transition penalty in time frame t. A penalty value σ is applied when switching between ascending and descending states to ensure that the algorithm avoids unnecessary state transitions. That is, Z(t)=0, if s(t)=s(t−1), and Z(t)=σ, if s(t)≠s(t−1). The objective function may be regarded as a penalized total path, which is calculated by summing up the penalized individual path V_{diff}(t)−s(t)−Z(t) of all time frames. And for calculating the penalized individual path at t=1, V(0) is defined as 0 for both s(0)=1 and s(0)=−1.

### Penalty Value

Penalty value (σ) may be any appropriate value to avoid unnecessary state transitions. In a preferred embodiment, a statistical measure which measures the spread or dispersion of the signal value is used. Examples of statistical measures which can be used as the penalty value include but not limited to interquartile range (IQR), standard deviation (SD), average absolute deviation (AAD), or median absolute deviation (MAD) of the wave signal. The definitions and key features of those statistical measures are described below.

### 1. Interquartile Range (IQR)

The definition of IQR is the range of the middle 50% of the data, i.e., the difference between the third quartile (Q3) and the first quartile (Q1), which has a formula: IQR=Q3−Q1. IQR is robust to outliers, and it focuses on the central portion of the data. However, it will ignore data in the tails.

### 2. Standard Deviation (SD)

The definition of SD is the square root of variance, measuring the average deviation from the mean in the same units as the data, which has a formula SD=\sqrt{Variance}. It has the same units as the data and is widely used. However, it is more sensitive to outliers.

### 3. Average Absolute Deviation (AAD)

The definition of AAD is the average of the absolute deviations from the mean, which has a formula

$$AAD = \frac{1}{n} {\sum }_{i = 1}^{n} \vert {x}_{i} - \overline{x} \vert .$$

Compared to variance or standard deviation, it is less sensitive to outliers than variance, but it is less common in statistical software and textbooks.

### 4. Median Absolute Deviation (MAD)

Median Absolute Deviation (MAD) is a robust measure, which is the median of the absolute deviations from the median of the data, and has a formula of MAD=median(|xᵢ−median(x)|). It is Highly robust to outliers, but it may not be sensitive enough for datasets with fine variability.

### State Determination

For a given signal V, the aim is to find a state series s(t) (for t=1 to T) which maximizes the objective value of the objective function

$$\begin{matrix}{\sum }_{t = 1}^{T} ( {V}_{diff} ( t ) . \\ s ( t ) - Z ( t ) ) .\end{matrix}$$

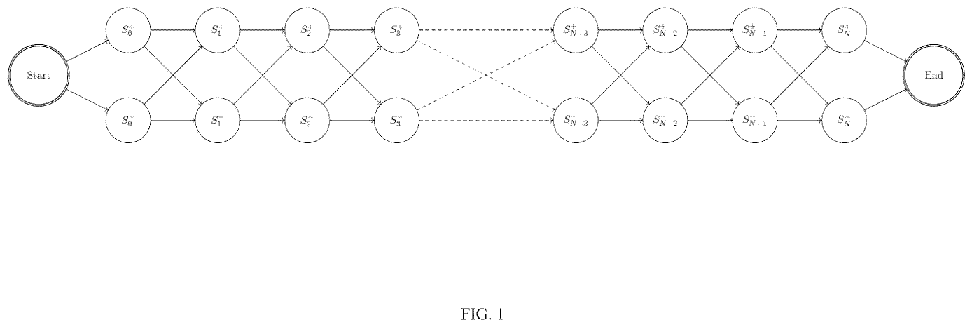

The maximal objective value is called an optimized final path. In each frame, the state s is either in ascending state (+1) or descending state (−1). The diagram shown in FIG. 1 illustrates possible state transitions of a wave signal. In short, in each time frame, the state s may be with the value +1 or −1.

If the state series

$${\{ s ( t ) \}}_{t = 1}^{T}$$

is determined, the objective value can be calculated by simply adding up penalized individual path P(t)=V_{diff}(t)·s(t)−Z(t) of all time frames. Since V_{diff}(t)=V(t)−V(t−1), and the value of Z(t) is determined by s(t) and s(t−1), it can be easily concluded that calculating the value of penalized individual path of the current time frame t only requires the information of V(t), V(t−1), s(t) and s(t−1). And because V(t) and V(t−1) are given values, and both s(t) and s(t−1) have only two possible values (+1 or −1), it can be concluded that the penalized individual path P(t) can only have four possible values, which are V_{diff}(t), V_{diff}(t)−σ, −V_{diff}(t), and −V_{diff}(t)−σ.

Now we define a penalized temporal path:

$$TP ( {t}^{′} , {s}^{′} ) = {\sum }_{t = 1}^{t′} ( {V}_{diff} ( t ) \cdot s ( t ) - Z ( t ) ) , wherein s ( {t}^{′} ) = {s}^{′} . \qquad ( Eq . 2 )$$

The penalized temporal path is the penalized path calculated from t=1 to t=t′ with a state value s′ at time frame t′. If t′ is the time frame we currently focus on, then s′ is the current state value. Among all possible TP(t′,s′), the maximum TP value with a current state value s′ at current time frame t′ is called current path and denoted as TP_{Max}(t′,s′) or DP(t′,s′). The current path with current state s′=1 is called first current path, and the other one with current state s′=−1 is called second current path.

By definition, the penalized temporal path of the current time frame t equals the penalized temporal path of the previous time frame t−1 (which is called previous temporal path) plus the penalized individual path of the current time frame t. That is, TP(t−1,s′)+V_{diff}(t)− s(t)− Z(t)=TP(t,s). Here, s′ is the state value of previous time frame t−1, which is called previous state value. We define the maximum TP value of previous time frame TP_{Max}(t−1,s′) as previous path, wherein the one with previous state s′=1 is called first previous path, and the other one with previous state s′=−1 is called second previous path. In other words, the first previous path is the largest among the previous temporal path having a status of ascending at the previous time frame t−1, and the second previous path is the largest among the previous temporal path having a status of descending at the previous time frame t−1. With the above equation, we have a recurrence relation:

$${TP}_{Max} ( t , s ) = {max}_{{s}^{′} ∈ \{ - 1 , 1 \}} [ {TP}_{Max} ( t - 1 , {s}^{′} ) + {V}_{diff} ( t ) \cdot s - Z ( t ) ] . \qquad ( Eq . 3 )$$

This means that TP_{Max}(t,1) is the larger one of first ascending path TP_{Max}(t−1,1)+V_{diff}(t) and second ascending path TP_{Max}(t−1,−1)+V_{diff}(t)−σ. Similarly, TP_{Max}(t,−1) is the larger one of first descending path TP_{Max}(t−1,1)−V_{diff}(t)−σ and second descending path TP_{Max}(t−1,−1)−V_{diff}(t). Using the recurrence relation of Eq. 3, TP_{Max}(t,1) and TP_{Max}(t,−1) can further be used to calculate TP_{Max}(t+1,1) and TP_{Max}(t+1,−1).

Based on the above analysis, it shows that TP_{Max}(t,s) for all t can be calculated, including TP_{Max}(T,s). And since the optimized final path is the largest among all possible objective values, it can be determined by selecting the larger one of TP_{Max}(T,1) and TP_{Max}(T,−1).

It is known by Eq. 3 that each TP_{Max}(t,s) corresponds to a TP_{Max}(t−1,s′) selected from TP_{Max}(t−1,1) and TP_{Max}(t−1,−1). Therefore, if we record the values of first previous path TP_{Max}(t−1,1) and second previous path TP_{Max}(t−1,−1), we can get the previous state value s′ at time frame t−1 which yields TP_{Max}(t,s). In other words, after calculating TP_{Max}(t,s), two previous state values can be determined, which are the previous state value corresponding to TP_{Max}(t,1), and the previous state value corresponding to TP_{Max}(t,−1). Starting from the optimized final path and its corresponding state value s(T), we can trace back previous state values all along to t=1, and the resulting state series {s(t)}ₜ₌₁^{T} is the optimized state series, which maximize the objective value.

The optimized state series may also be obtained via an alternative approach. Based on Eq. 3, it is known that each current path TP_{Max}(t,s) corresponds to a previous path TP_{Max}(t−1,s′) selected from the first previous path TP_{Max}(t− 1,1) and the second previous path TP_{Max}(t−1,−1). Therefore, the state series may be recorded and updated sequentially from the first time frame to the last time frame. In this approach, the calculated states of previous time frames are recorded as a first previous state series and a second previous state series, and the first previous state series and the second previous state series are updated with newly calculated results in every time frame. In particular, the first previous state series may be a state series {s}₁^{t-1} corresponding to the first previous path TP_{Max}(t− 1,1), and the second previous state series may be a state series {s}₁^{t-1} corresponding to the second previous path TP_{Max}(t−1,−1).

The optimized state series may be determined as follows. In each time frame, the first previous path TP_{Max}(t−1, 1), the first previous state series, the second previous path TP_{Max}(t−1,−1), the second previous state series, and the current displacement V_{diff}(t) are obtained. Then the first current path TP_{Max}(t,1) and the second current path TP_{Max}(t,−1) are calculated based on the first previous path, the second previous path, and the current displacement. As described above, this may be done by selecting the larger one of the first ascending path TP_{Max}(t−1,1)+V_{diff}(t) and the second ascending path TP_{Max}(t−1,−1)+V_{diff}(t)−σ as the first current path TP_{Max}(t,1), and selecting the larger one of the first descending path TP_{Max}(t−1,1)−V_{diff}(t)− σ and the second descending path TP_{Max}(t−1,−1)−V_{diff}(t) as the second current path TP_{Max}(t,−1). Next, the first previous path and the second previous path are respectively replaced by the first current path and the second current path for use in the next iteration.

Lastly, based on the selections which yield the first and second current paths, a first current state series corresponding to the first current path and a second current state series corresponding to the second current path can be obtained. For example, if the first current path is the first ascending path, then an ascending state is added to the first previous state series as the first current state series; if the first current path is the second ascending path, then an ascending sate is added to the second previous state series as the first current state series; if the second current path is the first descending path, then a descending state is added to the first previous state series as the second current state series; and if the second current path is the second descending path, then a descending state is added to the second previous state series as the second current state series. Then, the first previous state series and the second previous state series are also respectively replaced by the first current state series and the second current state series for use in the next iteration.

The above steps are repeated until the last time frame. Then, the larger of the first current path and the second current path at the last time frame is selected as an optimized final path, and the total state series corresponding to the optimized final path is selected as the optimized state series.

### Dynamic Programming

A dynamic programming algorithm can be used to perform the above objective value calculation and state series determination tasks. As described above

$${TP}_{Max} ( t , s ) = {max}_{{s}^{′} ∈ \{ - 1 , 1 \}} [ {TP}_{Max} ( t - 1 , {s}^{′} ) + {V}_{diff} ( t ) \cdot s - Z ( t ) ] ,$$

wherein Z(t)=0, if s(t)=s(t−1), and Z(t)=σ, if s(t)≠s(t−1). To calculate the value of TP(1,s), TP_{Max}(0,s′) is set to 0 for both s′=1 and s′=−1.

A pseudocode snippet performing dynamic programming (DP) method of objective value calculation and state series determination is as shown below. Here we use DP(t,s) to represent TP_{Max}(t,s) calculated by dynamic programming (DP) algorithm.

```
  $1 : Input : Sequences {\{ V ( t ) \}}_{t = 1}^{T} , σ$
  $2 : Output : Optimal state sequence {\{ s ( t ) \}}_{t = 1}^{T} and maximum objective value$
  3: Compute V_{diff}(t) = V(t) − V(t − 1) for t = 1, ... ,T (assume V(0) = 0 or given)
  4: Initialize DP(0, 1) = 0 and DP(0, −1) = 0
  5: for t = 1 to T do
  6: for each s in {1, −1} do
  7: bestV al ← − ∞
  8: for each s′ in {1, −1} do
  9: transitionObj ← V_{diff}(t) · s
10: if s ≠ s′ then
11: transitionObj ← transitionObj − σ
12: end if
13: if DP(t − 1, s′) + transitionObj > bestVal then
14: bestVal ← DP(t − 1, s′) + transitionObj
15: prevState(t, s) ← s′
16: end if
17: end for
18: DP(t, s) ← bestVal
19: end for
20: end for
21: maxObj ← max(DP(T, 1), DP(T, −1))
22: s(T) ← arg max_{s∈{1,-1}} DP(T, s)
23: for t = T−1 down to 1 do
24: s(t) ← prevState(t + 1, s(t + 1)
25: end for
$26 : return {\{ s ( t ) \}}_{t = 1}^{T} , maxObj$
```

### Implementation Examples

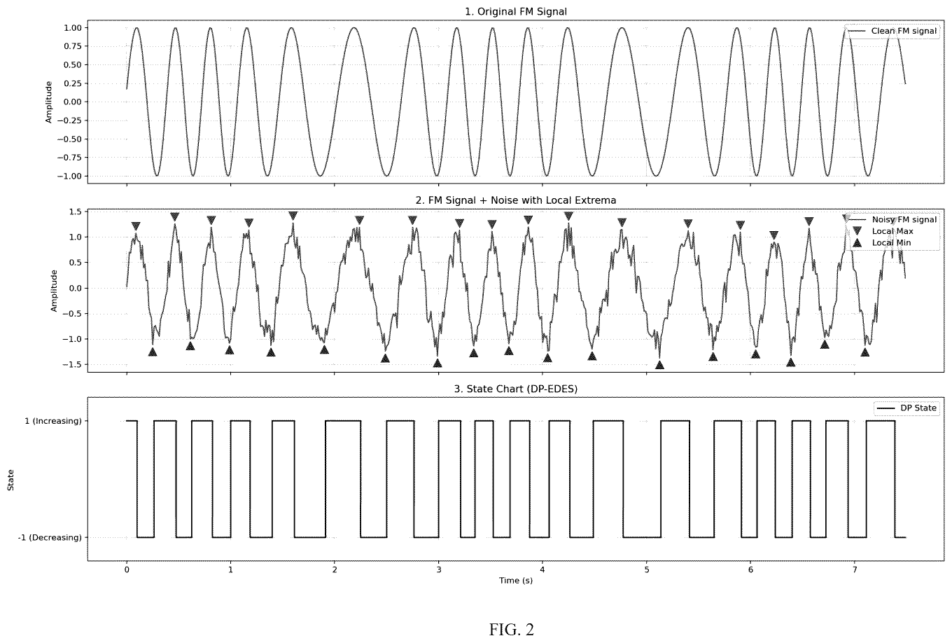

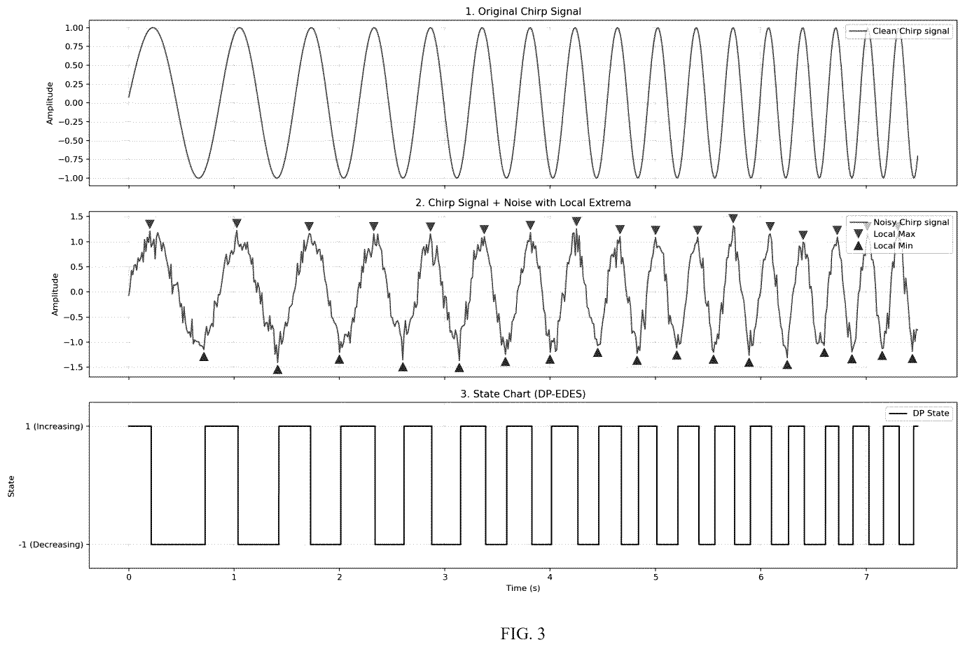

The computer-implemented method as described above addresses the limitations in existing methods for identifying local maxima and minima in quasi-cyclic time series, particularly in applications like heart signal analysis. FIGS. 2-3 are two exemplary wave signals for the DP algorithm to detect the phase transitions.

FIG. 2 shows an example of frequency modulated (FM) signal. The top row is an original frequency modulated (FM) signal, and some noises are introduced into the original signal, as shown in the middle row. Then a DP algorithm using the standard deviation of the wave signal as the penalty value is applied on the noise-introduced signal to detect transition points of the signal. The transition points detected by the algorithm are shown as dots on the curve in the middle row, and the phase (ascending/descending or on/off) changes are illustrated in the bottom row.

FIG. 3 shows a chirp signal, which is another kind of quasi-cyclic time series. The top row is an original chirp signal, and some noises are introduced into the original signal, as shown in the middle row. Likewise, a DP algorithm using the standard deviation of the wave signal as the penalty value is applied on the noise-introduced signal to detect transition points of the signal. The transition points detected by the algorithm are shown as dots on the curve in the middle row, and the phase changes are illustrated in the bottom row.

In echocardiography, The DP algorithm can be used to detect end-diastolic (ED) and end-systolic (ES) frame pairs from the left-ventricular (LV) endocardial length time series extracted from echocardiographic videos. This process is essential to track phases when the heart is most dilated (ED) and most contracted (ES).

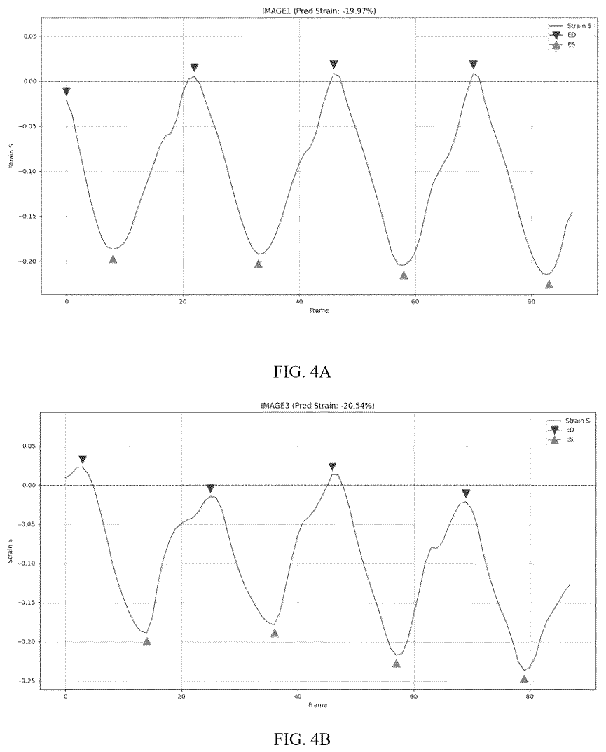

FIGS. 4A and 4B shows examples of how the DP algorithm is used to track heart muscle's contraction and relaxation cycle over time. The plots show myocardial strain versus time. The curve is the strain signal, where negative values mean the muscle is shortening or contracting. The algorithm find the extrema to pinpoint key cardiac events:
ES (End-Systole, ▾): Marks the troughs, which represent the moment of peak contraction.
ED (End-Diastole, ▴): Marks the peaks, representing the point of maximum relaxation just before contraction begins.

### Comparison to Previously Available Methods

Some methods for local extrema detection in quasi-cyclic time series are available. However, all those methods have their limitations. For example, traditional approaches often rely on localized comparisons, which can miss or inaccurately detect peaks in complex signals. The method of the present application detects globally optimal local maxima within each cycle of a quasi-cyclic time series, which ensures that all critical extrema are identified for complete data analysis. In addition, our method tolerates common signal deformations, such as level shifts, peak corruption, and valley shape variations. Previously available methods often fail under these condition, or require rounds of parameter tunning, whereas our method maintains accuracy without needing clean, well-defined peaks.

To give one real example, Scipy's find_peaks( ) function is a previously available method used by echonet's strain and unity's strain algorithm. It identifies local maxima (peaks) in a 1-D signal based on specified conditions. Users can filter peaks by properties like height, threshold, distance, prominence, width, and plateau size, and obtain both indices of peaks and a dictionary of peak properties. However, it requires much more parameters than our method. Below are the parameters required in Scipy's find_peaks( ) function.
(1) x: 1-D array of signal data to analyze for peaks.
(2) height: Required peak height or range [min, max].
(3) threshold: Vertical distance from neighbors, specified as a number or range [min, max].
(4) distance: Minimum sample distance between peaks; removes closer, smaller peaks.
(5) prominence: difference in height relative to surrounding baseline, specified as a number or range [min, max].
(6) width: Required peak width in samples; can be a number or range [min, max].
(7) wlen: Window length for prominence/width calculation, if provided.
(8) rel_height: Fraction of prominence used for width (default 0.5).
(9) plateau_size: Required flat top size of peaks in samples, as a number or range [min, max].

In comparison, our new method only needs a single parameter σ (penalty value) for transition points detection, and still shows a good extrema detection ability. Since the new method reduces the needs for parameter adjustments, it allows consistent performance across datasets. And because the new method does not require prior knowledge of the cycle period, it is suitable for signals with irregular cycles, such as frequency modulated signals, chirp signals and endocardial length time series as shown in the examples. The new method also shows high noise tolerance and jitter tolerance, which maintains accurate detection even with small noise disturbances and ensures correct extremum identification.

The foregoing description of embodiments is provided to enable any person skilled in the art to make and use the subject matter. Various modifications to these embodiments will be readily apparent to those skilled in the art, and the novel principles and subject matter disclosed herein may be applied to other embodiments without the use of the innovative faculty. The claimed subject matter set forth in the claims is not intended to be limited to the embodiments shown herein but is to be accorded the widest scope consistent with the principles and novel features disclosed herein. It is contemplated that additional embodiments are within the spirit and true scope of the disclosed subject matter. Thus, it is intended that the present invention covers modifications and variations that come within the scope of the appended claims and their equivalents.

What is claimed is:

1. A computer-implemented method to identify turning points of a wave signal, comprising:
obtaining a displacement series comprising displacement of each time frame of the wave signal;
based on the displacement series, determining a total state series which maximizes a penalized total path as an optimized state series; and
determining positions of turning points of the wave signal based on the optimized state series; wherein:
the total state series comprises a state value describing a status of ascending or descending of the wave signal at each time frame;
the penalized total path is calculated based on the displacement series and the total state series, wherein a penalty value is introduced to the penalized total path every time when the state value changes in the total state series.

2. The method of claim 1, wherein the penalty value is a standard deviation of the wave signal.

3. The method of claim 1, wherein the penalty value is an interquartile range of the wave signal.

4. The method of claim 1, wherein the penalty value is a mean absolute deviation of the wave signal.

5. The method of claim 1, wherein the penalty value is a median absolute deviation of the wave signal.

6. The method of claim 1, wherein the turning points of the wave signal are a plurality of time frames where the state value changes in the optimized state series.

7. The method of claim 1, wherein the penalized total path is calculated with a formula
$${\sum }_{t = 1}^{T} ( {V}_{diff} ( t ) \cdot s ( t ) - Z ( t ) ) ,$$
wherein:
T is a final time frame of the wave signal;
V_{diff}(t) is a current displacement, which is a value of the displacement series at time frame t;
s(t) is a current state value, which is a value of the total state series at time frame t, wherein the current state value is either 1 to represent a status of ascending or −1 to represent a status of descending; and
Z(t) is a transition penalty at time frame t, wherein Z(t)=the penalty value, if s(t)≠s(t−1) and Z(t)=0, if s(t)=s(t−1).

8. The method of claim 1, wherein the optimized state series is determined by a dynamic programming algorithm.

9. The method of claim 1, wherein said determining the optimized state series comprises iteratively calculating one or more penalized temporal paths and identifying one or more sub-optimal temporal paths from the one or more penalized temporal paths at each time frame.

10. The method of claim 9, wherein each of the one or more penalized temporal paths is calculated by adding a penalized individual path of a current time frame to a previous temporal path of a previous time frame.

11. The method of claim 10, wherein the penalized individual path comprises a current displacement and a transition penalty.

12. The method of claim 11, wherein each of the one or more penalized temporal paths are calculated based on a formula TP(t,s)=TP(t−1,s′)+P(t), wherein:
TP(t,s) is the penalized temporal path with a current state value s;
TP(t−1,s′) is the previous temporal path with a previous state value s′;
P(t)=V_{diff}(t)− s(t)− Z(t) is the penalized individual path, wherein:
V_{diff}(t) is the current displacement, which is a value of the displacement series at the current time frame t;
s(t) is the current state value, which is a value of the total state series at the current time frame t, wherein the current state value is either 1 to represent a status of ascending or −1 to represent a status of descending; and;
Z(t) is the transition penalty at time frame t, wherein Z(t)=the penalty value, if s(t)≠s(t−1) and Z(t)=0, if s(t)=s(t−1).

13. The method of claim 11, wherein the one or more sub-optimal temporal paths comprise a first current path with a status of ascending at the current time frame, and a second current path with a status of descending at the current time frame.

14. The method of claim 13, wherein the first current path and the second current path are calculated based on a first previous path, a second previous path and the penalized individual path; wherein:
the first previous path is the largest among the previous temporal path having a status of ascending at the previous time frame; and
the second previous path is the largest among the previous temporal path having a status of descending at the previous time frame.

15. The method of claim 14, wherein:
the first previous path corresponds to a first previous state series comprising a state value describing a status of ascending or descending of the wave signal at each time frame from a first time frame to the previous time frame, and the second previous path corresponds to a second previous state series comprising a state value describing a status of ascending or descending of the wave signal at each time frame from the first time frame to the previous time frame.

16. The method of claim 15, wherein the first previous path equals to the previous temporal path of the previous time frame calculated based on the displacement series and the first previous state series, and the second previous path equals to the previous temporal path of the previous time frame calculated based on the displacement series and the second previous state series.

17. The method of claim 15, wherein said determining the optimized state series comprises iterative steps of:
for each time frame, obtaining the first previous path, the first previous state series, the second previous path, the second previous state series, and the current displacement;
calculating the first current path and the second current path based on the first previous path, the second previous path, and the current displacement;
updating the first previous path with the first current path, and updating the second previous path with the second current path; and
updating the first previous state series with a first current state series corresponding to the first current path, and updating the second previous state series with a second current state series corresponding to the second current path.

18. The method of claim 17, wherein the first current path is the larger of a first ascending path and a second ascending path, and the second current path is the larger of a first descending path and a second descending path; wherein:
the first ascending path is the first previous path plus the current displacement;
the second ascending path is the second previous path plus the current displacement and minus the penalty value;
the first descending path is the first previous path minus the current displacement and the penalty value; and
the second descending path is the second previous path minus the current displacement.

19. The method of claim 18, wherein:
if the first current path is the first ascending path, then an ascending state is added to the first previous state series as the first current state series;
if the first current path is the second ascending path, then an ascending sate is added to the second previous state series as the first current state series;
if the second current path is the first descending path, then a descending state is added to the first previous state series as the second current state series; and
if the second current path is the second descending path, then a descending state is added to the second previous state series as the second current state series.

20. The method of claim 17, wherein at a final time frame of the wave signal further comprising steps of:
selecting the larger of the first current path and the second current path as an optimized final path; and
selecting the total state series corresponding to the optimized final path as the optimized state series.

21. The method of claim 14, wherein said determining the optimized state series comprises iterative steps of:
for each time frame, obtaining the first previous path, the second previous path, and the current displacement;
calculating the first current path and the second current path based on the first previous path, the second previous path, and the current displacement;
recording a first previous state value corresponding to the first current path;
recording a second previous state value corresponding to the second current path;
updating the first previous path with the first current path for use in next iterative step; and
updating the second previous path with the second current path for use in next iterative step.

22. The method of claim 21, after calculating the first current path and the second current path at a final time frame further comprising:
selecting the larger of the first current path and the second current path as an optimized final path;
iteratively determining a previous state value from the final time frame to a first time frame, wherein the previous state value at each time frame is selected from the first previous state value and the second previous state value; and
obtaining the previous state value at each time frame as the optimized state series.
